(19) United States
(12) Patent Application Publication
(10) Pub. No.: US 2018/0344648 A1
(43) Pub. Date: Dec. 6, 2018

(54) CLOBAZAM TABLET FORMULATION AND PROCESS FOR ITS PREPARATION

(71) Applicant: PIRAMAL ENTERPRISES LIMITED, Mumbai (IN)

(72) Inventors: Vipan DHALL, Ahmedabad, Gujarat (IN); Aravind KERUDI, Ahmedabad, Gujarat (IN); Tapan BUCH, Ahmedabad, Gujarat (IN); Ajay SAV, Ahmedabad, Gujarat (IN)

(73) Assignee: PIRAMAL ENTERPRISES LIMITED, Mumbai (IN)

(21) Appl. No.: 15/777,952

(22) PCT Filed: Nov. 29, 2016
(86) PCT No.: PCT/IB2016/057182

§ 371 Date: May 22, 2018

(30) Foreign Application Priority Data
Nov. 30, 2015 (IN) 4490/MUM/2015

(51) Int. Cl. A61K 9/20; A61K 31/5513

(52) U.S. Cl. CPC A61K 9/2077; A61K 31/5513; A61K 9/2013; A61K 9/2059; A61K 9/2009; A61K 9/2018

(57) ABSTRACT

A pharmaceutical composition comprising clobazam or its pharmaceutically acceptable salt(s) or solvate(s) thereof of defined particle size and one or more pharmaceutically acceptable excipients is described. A method for preparing the composition is also described.

### FIELD OF THE INVENTION

The present invention relates to a pharmaceutical composition comprising clobazam or its pharmaceutically acceptable salt)s) or solvate(s) thereof of defined particle size and one or more pharmaceutical excipient.

### BACKGROUND OF THE INVENTION

Clobazam is a 1,5-benzodiazepine derivative with antiepileptic and anti-psychotic

properties. The chemical name of Clobazam is 7-Chloro-1-methyl-5-phenyl-1,5-benzodiazepine-2,4(3H)-dione. Its molecular formula is C₁₆H₁₃O₂N₂Cl and the molecular weight is 300.7. The chemical structure is

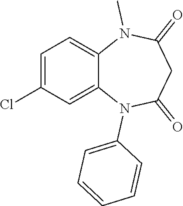 [SMILES: CN1C(=O)CC(=O)N(c2ccccc2)c2cc(Cl)ccc21]

Clobazam is administrated in the form of tablets that are marketed in the USA under the name ONFI® and the Europe under the name FRISIUM® for adjunctive treatment of seizures associated with Lennox-Gastaut syndrome (LGS) in patients two years and older.

Each ONFI® tablet contains 10 mg or 20 mg of clobazam and other ingredients such as corn starch (starch), lactose monohydrate, magnesium stearate, silicon dioxide, and talc. ONFI® is also available for oral administration as an off-white suspension containing clobazam at a concentration of 2.5 mg/mL and other ingredients include magnesium aluminum silicate, xanthan gum, citric acid monohydrate, disodium hydrogen phosphate dihydrate, simethicone emulsion, polysorbate 80, methylparaben, propylparaben, propylene glycol, sucralose, maltitol solution, berry flavor, purified water.

U.S. Pat. No. 4,721,709 discloses a pharmaceutical composition for oral administration comprising a hydrophobic, practically water-insoluble benzodiazepene drug. FR2531866 discloses tablets with rapid disintegration comprising one or more active compounds from the series of benzodiazepine derivatives or their therapeutically equivalent.

Formulations produced by using only corn starch do not have sufficient hardness and leads to capping when they are compressed into tablets under high pressure with intent to achieve sufficient hardness. The tablets which do not have sufficient hardness are subjected to breakage or crumble in blister packages. This is an undesired result in terms of pharmaceutical acceptability (Disintegrants—A Brief Review, Harish Gopinath et al).

Further, in pharmaceutical products, the particle size of drugs and excipients affects their processing and bioavailability. The effect of particle size of active pharmaceutical ingredient is related to dissolution, solubility, bioavailability, content uniformity, stability, product appearance and handling. Particle size reduction resulting in an increased surface area is a very promising approach to enhance dissolution rate and, consequently, the bioavailability of poorly water soluble drugs, such as clobazam. Arriving at a particle size distribution suitable to meet all desired criteria of a drug product is challenging and requires extensive investigation.

There exists an absolute need for the development of an improved formulation that would prevent capping and have sufficient hardness, yet increase dissolution of clobazam. Moreover, there is a need to provide a simpler and economically viable process of manufacturing, thus enabling overall cost effective manufacturing of clobazam product.

In consideration of the need as indicated above, inventors of the present invention directed their efforts to provide a pharmaceutical composition comprising clobazam or its pharmaceutically acceptable salt(s) or solvate(s) thereof of defined particle size and one or more pharmaceutical excipient and a simple and cost-effective process for making the same.

### SUMMARY OF THE INVENTION

In one aspect, the present invention relates to a pharmaceutical composition comprising clobazam or its pharmaceutically acceptable salt(s) or solvate(s) thereof of defined particle size and one or more pharmaceutical excipient.

In another aspect, the present invention relates to a process for the preparation of a pharmaceutical composition comprising clobazam or its pharmaceutically acceptable salt(s) or solvate(s) thereof of defined particle size and one or more pharmaceutical excipient.

In another aspect, there is provided a pharmaceutical composition of clobazam or its pharmaceutically acceptable salts or solvates thereof of defined particle size comprising pregelatinized starch along with one or more other pharmaceutical excipient. The advantages of using pregelatinized starch in the clobazam formulation are: 1. Simple scale up process, 2. Good physical properties that include good flow properties, 3. Avoids starch paste preparation thereby saving time, energy and other variations.

In another aspect, there is provided a pharmaceutical composition comprising clobazam with a defined particle size distribution and pregelatinized starch.

In another aspect, the present invention relates to a pharmaceutical composition comprising a) a therapeutically effective amount of clobazam or its pharmaceutically acceptable salt(s) or solvate(s) of defined particle size; b) from about 1% to about 30% by weight of pregelatinized starch; c) from about 40% to about 75% by weight of lactose monohydrate wherein the percentage by weight is relative to total weight of the composition thereof and one or more pharmaceutically acceptable excipient.

In another further aspect, there is provided a method for treating tonic-clonic, complex partial or myoclonic seizures; seizures associated with Lennox-Gastaut Syndrome; epilepsy or anxiety comprising administering to a subject in need thereof an effective amount of any one of the formulations of the present invention.

In another further aspect, there is provided use of a pharmaceutical composition comprising clobazam or its pharmaceutically acceptable salt(s) or solvate(s) thereof and one or more pharmaceutical excipient, for the manufacture of a medicament for treating tonic-clonic, complex partial or myoclonic seizures; seizures associated with Lennox-Gastaut Syndrome; epilepsy or anxiety.

In a still further aspect, the present invention relates to a pharmaceutical kit comprising: (a) clobazam or its pharmaceutically acceptable salt(s) or solvate(s) thereof and one or more pharmaceutical excipient; and (b) optionally a package insert comprising instructions for using the said pharmaceutical formulation.

These and other aspects and advantages of the present invention will be apparent to those skilled in the art from the following description.

### BRIEF DESCRIPTION OF THE DRAWINGS OF THE INVENTION

FIG. 1 represents dissolution profile of examples 1 to 6 with different particle size of clobazam.

FIG. 2 represents dissolution profile of examples 10 to 15 with different ratio of starch and Lactose monohydrate.

### DETAILED DESCRIPTION OF THE INVENTION

It should be understood that the detailed description and specific examples, while indicating embodiments of the invention, are given by way of illustration only, since various changes and modifications within the spirit and scope of the invention will become apparent to those skilled in the art. One skilled in the art, based upon the definitions herein, may utilize the present invention to its fullest extent. The following specific embodiments are to be construed as merely illustrative, and not limitative of the remainder of the disclosure in any way whatsoever.

Except, as defined herein, all the technical and scientific terms used herein have the same meaning as commonly understood by one of ordinary skill in the art to which the invention relates.

### Definitions

For the purpose of the disclosure, listed below are definitions of various terms used to describe the present invention. Unless otherwise indicated, these definitions apply to the terms as they are used throughout the specification and the appended claims, either individually or as part of a larger group. They should not be interpreted in the literal sense. They are not general definitions and are relevant only for this application.

It should be noted that, as used in this specification and the appended claims, the singular forms “a,” “an,” and “the” include plural referents unless the content clearly dictates otherwise.

It should be noted that the term “or” is generally employed in its sense including “and/or” unless the content clearly dictates otherwise.

As used herein, the term “about” means approximately and in the context of numerical values the terra “about” can be construed to estimate a value that is ±10% of the value or range recited.

The term “excipient” as used herein means a diluent, disintegrant, filler, glidant, lubricant, binder or the like, which is non-toxic, and inert, which does not have undesirable effects on a subject to whom it is administered and is suitable for delivering a therapeutically active agent (clobazam) to the target site without affecting the therapeutic activity of the said agent.

The term “pharmaceutically acceptable salt(s)” means salt(s) of clobazam, which can be prepared by treating clobazam with an appropriate acid or a base. Examples of pharmaceutically acceptable base addition salts include, but are not limited to, sodium, potassium, calcium, magnesium, ammonium salts or an organic base salt. Examples of pharmaceutically acceptable organic base addition salts include, but are not limited to, those derived from organic bases such as lysine, arginine, guanidine, and the like. Examples of pharmaceutically acceptable acid addition salts include, but are not limited to, those derived from inorganic acids such as hydrochloric acid, nitric acid, phosphoric acid, sulfuric acid and the like, as well as the salts derived from organic acids such as acetic acid, trifluoroacetic acid, propionic acid, oxalic acid, maleic acid, benzoic acid, succinic acid, fumaric acid, phthalic acid, benzenesulfonic acid, p-toluenesulfonic acid, citric acid, tartaric acid, methanesulfonic acid and the like.

Within the context of the present invention and as used herein, the term “solvate” or “solvates” describe a complex wherein the clobazam of the present invention, is coordinated with a proportional amount of a solvent molecule. Specific solvates, wherein the solvent is water, are referred to as hydrates.

As used herein, the term “formulation” or “composition” or “pharmaceutical composition” or “dosage form” as used herein synonymously include solid dosage forms such as granules, multiunit particulate systems (MUPS), pellets, spheres, tablets, capsules, mini-tablets, layered tablets, beads, particles and the like; and liquid dosage forms such as solutions, suspensions, emulsions, colloids and the like, meant for oral administration. The active pharmaceutical compound is clobazam.

The term “particle size” unless indicated otherwise in the specification relates to particles of clobazam as well as pharmaceutically acceptable salt or solvates thereof. Clobazam with specific “particle size” and distribution, or surface area would provide a fast dissolution of the active ingredient, would be easy to prepare and stable while maintaining the beneficial properties with respect to fast dissolution and bioavailability. Particularly, according to the present invention, clobazam having particle size less than about 170 microns are useful.

Within the context of the present invention and as used herein the term “clobazam” unless indicated otherwise in the entire specification, refers to clobazam in its free form, or as a pharmaceutically acceptable salt or solvates thereof.

Within the context of the present invention and as used herein, the term “clobazam particles of defined size” unless indicated otherwise in the entire specification, refers to the particle size (D₅₀) of clobazam or its pharmaceutically acceptable salts or solvates, which is between approximately 2μ and approximately 55μ. The particle size (D₉₀) of said clobazam or its pharmaceutically acceptable salts or solvates is between approximately 5μ and approximately 170μ.

Within the context of the present invention and as used herein, unless indicated otherwise, references to total weight of the pharmaceutical composition refers to the total weight of the active agent(s) and pharmaceutically acceptable excipient(s).

Within the context of the present invention and as used herein the term “subject” refers to an animal, preferably a mammal, and most preferably a human. In the context of the present invention, the term “mammal” is used interchangeably with the term “patient” or “subject”. In the context of the present invention, the phrase “a subject in need thereof” means a subject (patient) in need of the treatment of a disease or disorder for which clobazam is used.

Within the context of the present invention and as used herein the term ‘filler’ and the term ‘diluent’ are herein used interchangeably. Fillers fill out the size of a composition, making it practical to produce and convenient for the consumer to use. Suitable filler/diluent includes, but are not limited, to calcium carbonate, calcium phosphate, dibasic calcium phosphate, tribasic calcium sulfate, calcium carboxymethylcellulose, cellulose, dextrin derivatives, dextrin, dextrose, fructose, lactitol, lactose monohydrate, lactose (e.g. spray-dried lactose, α-lactose, β-lactose, Tablettose®, various grades of Pharmatose®, Microtose® or Fast-Floc®), methylcellulose polymers such as, e.g., Methocel A®, Methocel A4C®, Methocel A 15C®, Metocel A4M®), hydroxyethylcellulose, hydroxypropylcellulose, L-hydroxypropylcellulose (low substituted), hydroxypropyl methylcellulose (HPMC) (e.g. Methocel E®, F and K, Metolose SH®P0 of Shin-Etsu, grades of Methocel F® and Metolose 65 SH®, the 4,000, 15,000 and 100,000 cps grades of Methocel K®; and the 4,000, 15,000, 39,000 and 100,000 grades of Metolose 90 SH®), sodium carboxymethylcellulose, carboxym ethylene, carboxymethylhydroxyethylcellulose and other cellulose derivatives, magnesium carbonate, magnesium oxide, maltitol, maltodextrins, maltose, sorbitol, starch, pregelatinized starch, sucrose, sugar, and xylitol, erythritol.

Glidants improve the flowability of the composition, Glidants are, but not limited to, colloidal silica, silicon dioxide, powdered cellulose, talc, tribasic calcium phosphate, mixtures thereof and the like.

Lubricant is particularly preferred when the composition is a tablet to improve the tabletting process. Lubricants prevent composition ingredients from clumping together and from sticking to the tablet punches or capsule filling machine and improve flowability of the composition mixture. Lubricants are, but not limited to sodium oleate, sodium stearate, sodium benzoate, sodium chloride, stearic acid, sodium stearyl fumarate, calcium stearate, magnesium stearate, magnesium lauryl sulfate, sodium stearyl fumarate, sucrose esters or fatty acid, zinc, polyethylene glycol, talc, mixtures thereof and the like.

Binders are dry powders or liquid which are added during wet granulation process to promote granules and cohesiveness. Binders are, but not limited to, cellulose and its derivatives including, ethyl cellulose, hydroxypropyl cellulose, hydroxypropyl methylcellulose, methylcellulose and hydroxyethyl cellulose, carboxymethyl cellulose, gelatin, liquid glucose, corn starch (starch), maize starch, pregelatinized starch, hydrocolloids, sugars, polyvinyl pyrrolidone, sodium alginate, acacia, alginic acid, tragacanth, xanthan, used either alone or combinations thereof.

Disintegrants are agents added to tablet formulations to promote the breakup of the tablet or capsule into smaller fragments in an aqueous environment thereby increasing the available surface area and promoting a more rapid release of the drug substance. Disintegrants are, but not limited to, crosslinked polymers; crosslinked polyvinylpyrrolidone (crospovidone), crosslinked sodium carboxymethyl cellulose (croscarmellose sodium), starch pregelatined starch, polacrilin potassium, sodium starch glycolate, microcrystalline used either alone or combinations thereof.

Solvents that can be used as binder medium are, but not limited to, isopropyl alcohol, acetone, methanol and the like, and can be used alone or in combination.

One or more of these excipients can be selected and used by the artisan having regard to the particular desired properties of the solid dosage form. The amount of each type of excipient employed, e.g. glidant, binder, filler or diluent and lubricant may vary within ranges conventional in the art.

Suitable pharmaceutical compositions include, but are not limited to, capsules, tablets, granules, powders and unit dose pockets. Preferably oral pharmaceutical formulation is tablet. The tablet can be coated or non-coated.

Coating agents are, but not limited to, sugars, hydroxypropyl methylcellulose, hydroxypropyl cellulose, methylcellulose, ethylcellulose, polyvinyl alcohol, sodium carboxylmethylcellulose, coatings based on methacrylic acid and its esters, such as Eudragit®, mixtures thereof and the like.

Pregelatinized starch is a directly compressible form of starch consisting of intact and partially hydrolyzed ruptured starch grains. Pregelatinized starch has multiple uses in formulations; as a binder, filler or disintegrate. In addition to this, it is well known that pregelatinized starch shows improved flowability and lubricity while retaining its disintegrant capability and moisture stability. The use of the pregelatinized starch in the formulation can also prevent adhesion of granules to the mold during the compressing process (Asian J. Pharm. Res. Vol 2, Issue 1, 30-39, 2012). The degree of starch gelatinization can be varied to obtain fully pregelatinized or partially pregelatinized starch. Partially pregelatinized starch have properties of both native and fully gelatinized starch and are useful as both a binder and disintegrant in tablet formulations whereas fully pregelatinized starch are used as binder.

In one aspect, the present invention relates to a pharmaceutical composition comprising clobazam or its pharmaceutically acceptable salt(s) or solvate(s) thereof with one or more pharmaceutical excipient.

In another aspect, the present invention relates to a pharmaceutical composition comprising clobazam of defined particle size or its pharmaceutically acceptable salt(s) or solvate(s) thereof with one or more pharmaceutical excipient.

In an embodiment, the particle size (D₉₀) of said clobazam or its pharmaceutically acceptable salts or solvates thereof is between approximately 5μ and approximately 170μ.

In an embodiment, the particle size (D₉₀) of said clobazam or its pharmaceutically acceptable salts or solvates thereof is between approximately 50μ and approximately 120μ.

In an embodiment, the particle size (D₅₀) of said clobazam or its pharmaceutically acceptable salts or solvates thereof is between approximately 2μ and approximately 55μ.

In an embodiment, the particle size (D₅₀) of said clobazam or its pharmaceutically acceptable salts or solvates thereof is between approximately 10μ and approximately 30μ.

In an embodiment, the particle size (D₅₀) of said clobazam or its pharmaceutically acceptable salts or solvates thereof is between approximately 15μ and approximately 30μ.

In an embodiment, the particle size (D₁₀) of said clobazam or its pharmaceutically acceptable salts or solvates thereof is between approximately 1μ and approximately 10μ .

In an embodiment, the pharmaceutical composition contains clobazam or its pharmaceutically acceptable salts or solvates thereof in the range of 5% w/w to 20% w/w of the composition.

In an embodiment, the pharmaceutical composition contains clobazam or its pharmaceutically acceptable salts or solvates thereof in the range of 5% w/w to 10% w/w of the composition.

In an embodiment, the pharmaceutical composition contains clobazam or its pharmaceutically acceptable salts or solvates is between 5 mg to 40 mg.

In another embodiment the present invention provides pharmaceutical composition comprising clobazam as an active agent(s) from about 5% w/w to about 20% w/w of the composition, have a particle size (D₅₀) in the range of 2μ, to 55μ, with one or more other pharmaceutically acceptable excipient(s).

In an embodiment, the pharmaceutically acceptable excipient is selected from the group consisting of diluents, fillers, lubricants, binders, glidants and combinations thereof.

In an embodiment, the filler or diluent is selected from the group consisting of lactose monohydrate, sucrose, dextrose, mannose, fructose, galactose sugar alcohols such as sorbitol, mannitol, erythritol, xylitol, lactitol, starlac, starch, pregelatinized starch, dibasic calcium phosphate, tribasic calcium phosphate, calcium carbonate, calcium sulfate, powdered cellulose, microcrystalline cellulose, silicified microcrystalline cellulose, magnesium carbonate, magnesium oxide, magnesium alumino metasilicate and the like used either alone or in combinations thereof. The diluent may be used in the range of about 70-90% by weight of the composition.

In another embodiment, the lubricant is selected from the group consisting of calcium stearate, glycerol behenate, magnesium stearate, mineral oil, polyethylene glycol, sodium stearyl fumarate, stearic acid, talc, vegetable oil, zinc stearate and the like used either alone or in combinations thereof. The lubricant may be used in the range of about 0.5-3% by weight of the composition.

In another embodiment, the glidant is selected from the group consisting of talc, silicon dioxide, starch and the like used either alone or in combination thereof. The glidant may be used in the range of about 0.5-3% by weight of the composition.

In an embodiment, the binder is selected from the group consisting of cellulose and its derivatives including, ethyl cellulose, hydroxypropyl cellulose, hydroxy-propyl methyl cellulose, methylcellulose and hydroxyethyl cellulose, carboxymethyl cellulose; gelatin, liquid glucose; starch; pregelatinized starch; hydrocolloids; sugars; polyvinyl pyrrolidone, sodium alginate, acacia, alginic acid, tragacanth, xanthan, used either alone or combinations thereof. The binder may be used in the range of about 1-20% by weight of the composition.

In another embodiment, the binder is selected from the group consisting of pregelatinized starch and starch or the combination thereof.

In an embodiment, the diluents are pregelatinized starch and lactose monohydrate.

In an embodiment, the starch and lactose monohydrate are in the range of about 1:1 to about 1:10.

In an embodiment, the starch and lactose monohydrate are in the range of about 1:1 to about 1:16.

In an embodiment, the starch and lactose monohydrate is 1:5.

In an embodiment, the lubricant is magnesium stearate.

In an embodiment, the glidants are talc and silicon dioxide.

In another embodiment, the composition comprises starch and/or pregelatinized starch.

In another embodiment, the pharmaceutical composition of the present invention comprises clobazam particles of defined size, lactose monohydrate, pregelatinized starch, starch, magnesium stearate, talc, silicon dioxide and combinations thereof.

In another embodiment, the composition of the invention can be in standard-release, immediate-release, rapid-onset, sustained-release or dual-release form.

In another embodiment, the composition of the invention is immediate-release form.

### Process for the Preparation of Formulation

In an aspect, the present invention relates to a process for preparing a pharmaceutical composition containing a therapeutically effective amount of clobazam or its pharmaceutically acceptable salts or solvates thereof comprising:

(i) micronizing said clobazam until the particle size (D₅₀) of said clobazam is equal to or less than about 55μ; and

(ii) combining said micronized clobazam or its pharmaceutically acceptable salts or solvates with one or more pharmaceutically acceptable excipient;

wherein said micronized clobazam or its pharmaceutically acceptable salts or solvates and said pharmaceutically acceptable excipient are combined by a wet or a dry granulation process.

In an embodiment, wet granulation process comprises:

(i) sifting said micronized clobazam or its pharmaceutically acceptable salts or solvates with at least one diluent, and other excipients to form a mixture;

(ii) granulating the mixture of step (i) with water or solvent;

(iii) drying and milling the granulated mixture of step (ii),

(iv) blending the mixture of step (iii) with a at least one filler and excipients;

(v) blending the mixture of step (iv) with at least one lubricant; and

(vi) compressing the drug mixture of step (v) into a pharmaceutical dosage.

In an embodiment, the dry granulation process comprises:

(i) clobazam particles with one or more pharmaceutically acceptable excipients are blended to form a first mixture. Preferably, clobazam particles are thoroughly dry blended with at least one glidant, diluent, binder and disintegrant to form a uniform mixture;

(ii) the resulting mixture is sized using an oscillator granulator or similar equipment used in the art for this purpose to produce granules and sieved to separate the desired size fraction. Preferably, the slugs of the invention are sized by passing through a 30# sieve mesh (0.595 mm size);

(iii) the granules are further blended with additional one or more pharmaceutically acceptable excipients to form a second mixture. Preferably, the granules are thoroughly dry blended with at least one glidant, diluent, binder and disintegrant, to form a uniform dry mixture;

(iv) the resulting mixture is sized using an oscillator granulator, or similar equipment used in the art for this purpose, fitted with a 30# sieve ( 0.595 mm size) mesh to produce granules;

(v) the granules from the resulting mixture is blended with an extragranular portion of one or more pharmaceutically acceptable excipients;

(vi) blending the mixture of step (v) with at least one lubricant; and

(vii) the drug mixture of step (v) is compressed into a final tablet;

(viii) optionally coating the tablets with a suitable coating material.

In an embodiment, the first mixture of the pharmaceutical composition of the present invention may comprise clobazam, lactose monohydrate or a combination thereof.

In another embodiment, the second mixture of the pharmaceutical composition of the present invention may comprise clobazam, lactose monohydrate, pregelatinized starch, starch and talc, or a combination thereof. Lubricants may be added in the final step to the granules obtained from the second mixture.

In another embodiment, the dry blend can be performed in a suitable mixer, such as a container blender, fluid bed dryer, drum blender, v-blender or a high shear mixer.

In an embodiment, tablet compression can be performed in a tablet press, and the optional coating process can be performed in a coating pan or fluid bed.

In another embodiment, the tablet formulations produced in the scope of the present invention can optionally be coated in order to provide various release characteristics, for instance fast release, sustained release, slow release or they are coated with film coating.

In yet another embodiment, the clobazam tablets of the present invention can be prepared by conventional tablet forming techniques such as, for example, wet granulation and dry granulation.

In the wet granulation process, the active ingredient is mixed with some or all of the filler. This blend is then wet granulated with water or an organic solvent, optionally containing a binder in solution. The resultant wet granulation is then dried and milled. The granules are then mixed with the remaining ingredients, which will include the lubricant, to produce the final mix, which is then compressed into tablets.

In the dry granulation process, the active ingredient is mixed with the other ingredients without addition of any solvent, and thus without the need for drying. Again the final mix is compressed into tablets.

The compositions of the present invention can be packed into suitable containers such as bottles, blisters or pouch. Further, the packages may optionally contain a desiccant or an antioxidant or oxygen absorbant or combinations thereof.

In an aspect, the present invention relates to use of the pharmaceutical composition comprising clobazam as a therapeutic agent, wherein the said formulation is as described herein above in one or more embodiments of the present invention.

In another embodiment, the present invention relates to a method of treating tonic-clonic, complex partial or myoclonic seizures; seizures associated with Lennox-Gastaut Syndrome; epilepsy or anxiety comprising administering to a subject in need thereof a therapeutically effective amount of the clobazam formulation; wherein the said formulation is as described in one or more embodiments of the present invention as described herein above.

In another embodiment, the present invention relates to use of the formulation of clobazam, for the manufacture of a medicament for treating or preventing tonic-clonic, complex partial or myoclonic seizures, seizures associated with Lennox-Gastaut Syndrome, epilepsy or anxiety; wherein the said formulation is as described herein above in one or more embodiments of the present invention.

In another embodiment, the formulation of clobazam; may be packaged in a suitable container depending upon the formulation and the method of administration of the composition. Suitable containers known to a person skilled in the art include blister pack or bottle pack.

In another embodiment, the present invention provides a pharmaceutical kit comprising clobazam or its pharmaceutically acceptable salt(s) or solvate(s) thereof; and one or more pharmaceutically acceptable excipient. The kit may further comprise a package insert, including information about the indication, usage, doses, direction for administration, contraindications, precautions and warnings.

In another embodiment, the pharmaceutical compositions of the present invention can include all the dosage forms known to a person skilled in art, viz. formulations such as single unit dosage forms in the form of tablets, bilayer tablets, inlaid tablets, tablet in tablet, multilayered tablets, minitablets filled in capsules and the like; beads, pellets presented in a sachet, capsule or tablet capsules such as soft and hard gelatin; lozenges or sachets; granulates, microparticles, multiparticulates, powder and the like.

It is understood that modifications that do not substantially affect the activity of the various embodiments of this invention are included within scope of the invention disclosed herein. Accordingly, the following examples are intended to illustrate but not to limit the scope of the present invention.

### EXAMPLES

As is appreciated by those in the art, different measurement instruments may provide different measurements of the same particles. Therefore, the result for the sizes obtained may not be exactly replicated between the different methods although it is expected that they will be relatively comparable. Clobazam is micronized using air jet mill. Applicants have therefore performed clobazam particle size analysis using Malvern Mastersizer 2000.

### Example A

### Particle Size Analysis for Clobazam Formulation

TABLE 1
Different formulations were prepared using following particle sizes:

| Batch no. | Example 1 | Example 2 | Example 3 | Example 4 | Example 5 | Example 6 | Example 7 | Example 8 |
|---|---|---|---|---|---|---|---|---|
| D₉₀ (μ) | 57 | 5.78 | 11.12 | 76.90 | 117.74 | 5.78 | 55.12 | 88 |
| D₅₀ (μ) | 14 | 2.61 | 3.92 | 16.26 | 23.35 | 2.61 | 12.01 | 31 |
| D₁₀ (μ) | 2 | 1.17 | 1.53 | 3.30 | 3.75 | 1.17 | 3.09 | 7 |

### Example B

### Summary of Dissolution Rates of Clobazam

TABLE 2A
Formulation compositions (with starch and without starch)

| | Batch No. | | | | | | | |
|---|---|---|---|---|---|---|---|---|
| | Example 1 | | Example 2 | | Example 3 | | Example 4 | |
| | D₉₀/D₅₀ (μ) | | | | | | | |
| | 57/14 | | 5.78/2.6 | | 11.1/3.92 | | 76.9/16.26 | |
| Ingredients | mg/tablet | % w/w | mg/tablet | % w/w | mg/tablet | % w/w | mg/tablet | % w/w |
| Clobazam | 20.0 | 8.33 | 20.0 | 8.33 | 20.0 | 8.33 | 20.0 | 8.33 |
| Lactose Monohydrate | 144.6 | 60.25 | 144.6 | 60.25 | 144.6 | 60.25 | 144.6 | 60.25 |
| Pregelatinized starch | 70.0 | 29.17 | 70.0 | 29.17 | 35.0 | 14.58 | 35.0 | 14.58 |
| Corn starch | — | — | — | — | 35.0 | 14.58 | 35.0 | 14.58 |
| Talc | 2.4 | 1.0 | 2.4 | 1.0 | 2.4 | 1.0 | 2.4 | 1.0 |
| Silicon dioxide | 0.6 | 0.25 | 0.6 | 0.25 | 0.6 | 0.25 | 0.6 | 0.25 |
| Magnesium Stearate | 2.4 | 1.0 | 2.4 | 1.0 | 2.4 | 1.0 | 2.4 | 1.0 |
| Purified Water | q.s. | 18.13 | q.s. | 24.32 | q.s. | q.s. | 24.43 | 24.43 |
| Total | 240.0 | 100.0 | 240.0 | 100.0 | 240.0 | 100.0 | 240.0 | 100.0 |

TABLE 2B
Formulation compositions (with starch and without starch)

| | Batch No. | | | | | | | |
|---|---|---|---|---|---|---|---|---|
| | Example 5 | | Example 6 | | Example 7 | | Example 8 | |
| | D₉₀/D₅₀ (μ) | | | | | | | |
| | 117.74/23.35 | | 5.78/2.6 | | 55.12/12.01 | | 88/31 | |
| Ingredients | mg/tablet | % w/w | mg/tablet | % w/w | mg/tablet | % w/w | mg/tablet | % w/w |
| Clobazam | 20.0 | 8.33 | 20.0 | 8.33 | 20.0 | 8.33 | 20.0 | 8.33 |
| Lactose Monohydrate | 144.6 | 60.25 | 144.6 | 60.25 | 144.6 | 60.25 | 144.6 | 60.25 |
| Pregelatinized starch | 35.0 | 14.58 | — | — | 35.0 | 14.58 | 35.0 | 14.58 |
| Corn starch | 35.0 | 14.58 | 70.0 | 29.17 | 35.0 | 14.58 | 35.0 | 14.58 |
| Talc | 2.4 | 1.0 | 2.4 | 1.0 | 2.4 | 1.0 | 2.4 | 1.00 |
| Silicon dioxide | 0.6 | 0.25 | 0.6 | 0.25 | 0.6 | 0.25 | 0.6 | 0.25 |
| Magnesium Stearate | 2.4 | 1.0 | 2.4 | 1.0 | 2.4 | 1.0 | 2.4 | 1.0 |
| Purified Water | q.s. | 24.43 | q.s. | 24.43 | q.s. | 24.43 | q.s. | 24.43 |
| Total | 240.0 | 100.0 | 240.0 | 100.0 | 240.0 | 100.0 | 240.0 | 100.0 |

### Example C

### Dissolution Study in Different Medium

TABLE 3A
Clobazam test tablet is released in 0.1N HCl environment under
conditions of 900 mL of a dissolution medium at 37° C. ±
0.5° C., USP method-II (paddle), 75 rpm (revolution per minute)
speed wherein the tablet exhibits a dissolution profile as follows:

| | Batch no. | | | | | | | |
|---|---|---|---|---|---|---|---|---|
| | Example 1 | | Example 2 | | Example 3 | | Example 4 | |
| Time | % | | % | | % | | % | |
| (min) | Mean | RSD | Mean | RSD | Mean | RSD | Mean | RSD |
| 0 | 0 | 0 | 0 | 0 | 0 | 0 | 0 | 0 |
| 5 | 37 | 3.1 | 63 | 0.9 | 62 | 2.5 | 35 | 1.7 |
| 10 | 50 | 2.0 | 80 | 1.9 | 82 | 1.9 | 50 | 1.2 |
| 15 | 59 | 1.7 | 87 | 1.8 | 91 | 1.7 | 60 | 2.6 |
| 20 | 66 | 2.3 | 90 | 1.7 | 94 | 1.6 | 64 | 1.8 |
| 30 | 74 | 2.8 | 94 | 1.1 | 96 | 1.8 | 73 | 2.1 |
| 45 | 83 | 2.1 | 96 | 1.0 | 97 | 1.6 | 80 | 2.6 |
| 60 | 88 | 3.0 | 97 | 1.0 | 97 | 1.6 | 86 | 3.1 |

TABLE 3B

| | Batch no. | | | | | | | |
|---|---|---|---|---|---|---|---|---|
| | Example 5 | | Example 6 | | Example 7 | | Example 8 | |
| Time | % | | % | | % | | % | |
| (minutes) | Mean | RSD | Mean | RSD | Mean | RSD | Mean | RSD |
| 0 | 0 | 0 | 0 | 0 | 0 | 0 | 0 | 0 |
| 5 | 23 | 2.5 | 63 | 14.5 | 30 | 0 | 24 | 0 |
| 10 | 34 | 1.7 | 86 | 8.4 | 48 | 2.1 | 41 | 1.4 |
| 15 | 41 | 1.4 | 92 | 4.5 | 59 | 1.7 | 51 | 0 |
| 20 | 47 | 1.2 | 95 | 0.6 | 67 | 1.5 | 58 | 1.0 |
| 30 | 54 | 1.1 | 96 | 0.6 | 77 | 1.3 | 68 | 1.5 |
| 45 | 63 | 1.6 | 96 | 1.2 | 85 | 1.2 | 77 | 0.8 |
| 60 | 69 | 0.0 | 96 | 0.6 | 91 | 1.7 | 82 | 1.2 |

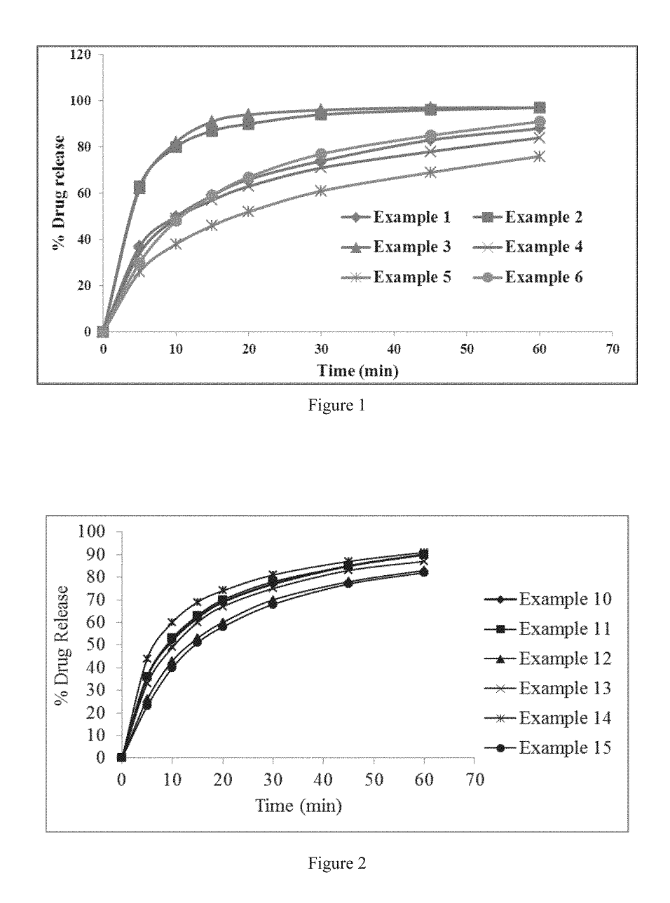

Summary: Dissolution study showed that Example 2 and Example 3, having particle size D₅₀ 2.61μ and 3.92μ respectively, have faster drug release (FIG. 1) and Example 5 exhibits slow drug release. Desired dissolution profile was obtained with Example 1, Example 4, Example 7 and Example 8.

TABLE 4
Clobazam test tablet is released in pH 6.8 phosphate buffer environments
under conditions of 900 mL of a dissolution medium at 37° C. ± 0.5° C.,
USP method-II (paddle), 50 rpm speed wherein the tablet exhibits a
dissolution profile as follows (RSD: relative standard deviation):

| Batch no. | |
|---|---|
| Example 5 | Example 7 |

| Time (min) | % Mean | RSD | % Mean | RSD |
|---|---|---|---|---|
| 0 | 0 | 0 | 0 | 0 |
| 5 | 21 | 0 | 23 | 2.5 |
| 10 | 32 | 0 | 41 | 1.4 |
| 15 | 38 | 1.5 | 53 | 1.1 |
| 20 | 43 | 1.3 | 62 | 1.6 |
| 30 | 49 | 2.0 | 73 | 1.6 |
| 45 | 55 | 2.8 | 83 | 1.8 |
| 60 | 60 | 2.6 | 90 | 2.2 |

Summary: Example 7, having particle size D₉₀ of 55μ showed faster drug release as compared to higher particle size as in Example 5, wherein D₉₀ is 117,74μ.

### Example D

### Effect of Binders in Clobazam Formulation

TABLE 5A
Different formulations were prepared with pregelatinized starch,
starch and combinations of both starch and pregelatinized starch.

| | Batch No. | | | | | | | | | |
|---|---|---|---|---|---|---|---|---|---|---|
| | Example 1 | | Example 2 | | Example 3 | | Example 4 | | Example 6 | |
| | D₉₀/D₅₀ (μ) | | | | | | | | | |
| | 57/14 | | 5.78/2.6 | | 11.1/3.92 | | 76.9/16.26 | | 40.67/11.36 | |
| Ingredients | mg/tablet | % w/w | mg/tablet | % w/w | mg/tablet | % w/w | mg/tablet | % w/w | mg/tablet | % w/w |
| Clobazam | 20.0 | 8.33 | 20.0 | 8.33 | 20.0 | 8.33 | 20.0 | 8.33 | 20.0 | 8.33 |
| Lactose Monohydrate | 144.6 | 60.25 | 146.6 | 60.25 | 144.6 | 60.25 | 144.6 | 60.25 | 144.6 | 60.25 |
| Pregelatinized starch | 70.0 | 29.17 | 70.0 | 29.17 | 35.0 | 14.58 | 35.0 | 14.58 | — | — |
| Corn starch | — | — | — | — | 35.0 | 14.58 | 35.0 | 14.58 | 70 | 29.17 |
| Talc | 2.4 | 1.0 | 2.4 | 1.0 | 2.4 | 1.0 | 2.4 | 1.0 | 2.4 | 1.0 |
| Silicon dioxide | 0.6 | 0.25 | 0.6 | 0.25 | 0.6 | 0.25 | 0.6 | 0.25 | 0.6 | 0.25 |
| Magnesium Stearate | 2.4 | 1.0 | 2.4 | 1.00 | 2.4 | 1.0 | 2.4 | 1.0 | 2.4 | 1.0 |
| Purified Water | q.s. | 18.13 | q.s. | 18.0 | q.s. | 24.32 | q.s. | 24.43 | q.s. | 24.43 |
| Total | 240.0 | 100.0 | 240.0 | 100.0 | 240.0 | 100.0 | 240.0 | 100.0 | 240.0 | 100.0 |

TABLE 6
Tablet formulation: All formulation compressed using oval tooling.

| | Example 1 | Example 2 | Example 3 | Example 6 | Example 4 |
|---|---|---|---|---|---|
| Hardness | 8-10 | 10-11 | 8-10 | 5-6.5 | 6-8 |
| (kp) | | | | | |
| Thickness | 4.45-4.46 | 4.51-4.53 | 4.47-4.50 | 4.28-4.36 | 4.38-4.40 |
| (mm) | | | | | |
| Disintegra- | 30-32 sec | 45-48 sec | 20-26 sec | 40-45 sec | 16-18 sec |
| tion Time | | | | | |

Table 6 indicates that the addition of pregelatinized starch showed better and improved physical properties such as flowability, thickness and hardness. It also devoid of tableting problems like capping or hardness issues as observed with starch alone.

### Example E

### Excipients Study

TABLE 7
Batches of Clobazam 20 mg Tablet with different ratio of Pregelatinized starch to lactose monohydrate

| Batch No. | Example 10 | Example 11 | Example 12 | Example 13 | Example 14 | Example 15 |
|---|---|---|---|---|---|---|
| Pregelatinized | 1:2.73 | 1:3.72 | 1:3.55 | 1:4.66 | 1:2.66 | 1:5.61 |
| starch:Lactose | | | | | | |
| monohydrate | | | | | | |
| Ingredients | % w/w | % w/w | % w/w | % w/w | % w/w | % w/w |
| Clobazam | 8.33 | 8.33 | 8.33 | 8.33 | 8.33 | 8.33 |
| Pregelatinized starch | 21.34 | 12.50 | 18.75 | 12.50 | 18.75 | 12.50 |
| Lactose Monohydrate | 58.33 | 46.55 | 66.67 | 58.33 | 50.00 | 70.12 |
| Corn Starch | 9.75 | 30.37 | 4.0 | 18.58 | 20.67 | 6.80 |
| Purified water | — | — | — | — | — | — |
| Talc | 1.0 | 1.0 | 1.0 | 1.0 | 1.0 | 1.0 |
| Colloidal Silicon | 0.25 | 0.25 | 0.25 | 0.25 | 0.25 | 0.25 |
| dioxide | | | | | | |
| Magnesium Stearate | 1.00 | 1.0 | 1.0 | 1.0 | 1.00 | 1.0 |
| Total | 100.0 | 100.0 | 100.0 | 100.0 | 100.0 | 100.0 |

Dissolution in 0.1N HCl, USP Type II, 75 rpm, 900 mL volume

| | Pregelatinized starch:Lactose monohydrate | | | | | |
|---|---|---|---|---|---|---|
| Dissolution | 1:2.73 | 1:3.72 | 1:3.55 | 1:4.66 | 1:2.66 | 1:5.61 |
| time point | % Drug | % Drug | % Drug | % Drug | % Drug | % Drug |
| (minutes) | release | release | release | release | release | release |
| 5 | 36 | 36 | 26 | 33 | 44 | 23 |
| 10 | 52 | 53 | 43 | 49 | 60 | 40 |
| 15 | 62 | 63 | 53 | 60 | 69 | 51 |
| 20 | 69 | 70 | 60 | 67 | 74 | 58 |
| 30 | 77 | 78 | 70 | 75 | 81 | 68 |
| 45 | 85 | 85 | 78 | 83 | 87 | 77 |
| 60 | 90 | 90 | 83 | 87 | 91 | 82 |

TABLE 8
Physical parameters of batches of Clobazam 20 mg Tablets with
different ratio of Pregelatinized starch to lactose monohydrate

| | | | | | | |
|---|---|---|---|---|---|---|
| Batch no. | Example 10 | Example 11 | Example 12 | Example 13 | Example 14 | Example 15 |

Lubricated Blend

| | | | | | | |
|---|---|---|---|---|---|---|
| Bulk density (g/mL) | 0.552 | 0.582 | 0.530 | 0.560 | 0.580 | 0.538 |

Particle size distribution (% cumulative weight retained)

| | | | | | | |
|---|---|---|---|---|---|---|
| 20# | 0.4 | 0.7 | 2.4 | 1.2 | 0.8 | 1.6 |
| 30# | 9.6 | 4.0 | 19.6 | 7.2 | 6.4 | 16.8 |
| 40# | 20.0 | 8.0 | 36.0 | 15.6 | 12.8 | 31.2 |
| 60# | 49.6 | 30.8 | 68.4 | 36.4 | 28.8 | 59.6 |
| 80# | 66.4 | 50.8 | 83.2 | 52.4 | 40.4 | 72.4 |
| 100# | 72.4 | 60.7 | 91.1 | 66.0 | 51.2 | 76.8 |
| Pan | 97.3 | 100.2 | 98.4 | 102.0 | 98.4 | 94.8 |

Compression

| | | | | | | |
|---|---|---|---|---|---|---|
| Hardness (kp) | 7-8 | 7-8 | 7-8 | 7-8 | 7-8 | 7-8 |
| Disintegrationtime | 1:00-1:15 | 0:15-0:20 | 6:00-6:30 | 0:60-0:80 | 0:20-0:25 | 6:00-7:00 |
| (min:sec) | | | | | | |
| Thickness (mm) | 4.20-4.35 | 4.25-4.30 | 4.19-4.31 | 4.19-4.25 | 4.30-4.36 | 4.20-4.30 |

Observation: Table 7 indicates that weight ratio ranging from about 1:1 to 1:6 of Pregelatinized starch to lactose monohydrate showed better and improved physical properties (FIG. 2) such as followability, thickness and hardness.

1. A pharmaceutical composition comprising a therapeutically effective amount of clobazam or its pharmaceutically acceptable salt(s) or solvate(s) thereof of defined particle size and one or more pharmaceutically acceptable excipient.

2. The pharmaceutical composition as claimed in claim 1, wherein the particle size of said clobazam or its pharmaceutically acceptable salts or solvates thereof is about 2 micron to about 170 micron.

3. The pharmaceutical composition as claimed in claim 1, wherein the particle size (D₅₀) of said clobazam or its pharmaceutically acceptable salts or solvates thereof is between approximately 2μ and approximately 55μ.

4. The pharmaceutical composition as claimed in claim 1, wherein the particle size (D₅₀) of said clobazam or its pharmaceutically acceptable salts or solvates thereof is between approximately 5μ and approximately 170μ.

5. The pharmaceutical composition as claimed in claim 1, wherein the quantity of said clobazam or its pharmaceutically acceptable salts or solvates is between 5 mg to 40 mg.

6. The pharmaceutical composition as claimed in claim 1, wherein the said excipient comprises of one or more of diluents, lubricants, fillers, binders, disintegrants, glidants, or a combination thereof.

7. The pharmaceutical composition as claimed in claim 1, wherein said pharmaceutically acceptable excipient is selected from the group consisting of pregelatinized starch, starch, sodium croscarmellose, crosslinked polyvinylpyrrolidone, talc, sodium lauryl sulfate, stearic acid, calcium stearate, magnesium stearate, microcrystalline cellulose, lactose monohydrate, starch, mannitol, potassium, chloride, powdered cellulose, sodium chloride, sorbitol hydroxyethyl cellulose, hydroxypropyl cellulose, hydroxypropyl methyl cellulose, colloidal silicon dioxide, croscarmellose sodium, crospovidone and methyl cellulose.

8. The pharmaceutical composition as claimed in claim 6, wherein the said diluent is selected from the group consisting of lactose monohydrate, sucrose, dextrose, mannose, fructose, galactose sugar-alcohols such as sorbitol, mannitol, erythritol, xylitol, lactitol, starlac, starch, pregelatinized starch, dibasic calcium phosphate, tribasic calcium phosphate, calcium carbonate, calcium sulfate, powdered cellulose, microcrystalline cellulose, silicified microcrystalline cellulose, magnesium carbonate, magnesium oxide, magnesium alumino metasilicate and/or a combination thereof.

9. The pharmaceutical composition as claimed in claim 8, wherein the said diluents is in the range of about 70-90% by weight of the composition.

10. The pharmaceutical composition as claimed in claim 8, wherein the diluents are pregelatinized starch and lactose monohydrate.

11. The pharmaceutical composition as claimed in claim 10, wherein the pregelatinized starch and lactose monohydrate are present in weight ratio from about 1:1 to about 1:10.

12. The pharmaceutical composition as claimed in claim 6, wherein the said lubricant is selected from the group consisting of calcium stearate, glycerol behenate, magnesium stearate, mineral oil, polyethylene glycol, sodium stearyl fumarate, stearic acid, talc, vegetable oil, zinc stearate and/or a combination thereof.

13. The pharmaceutical composition as claimed in claim 12, wherein the said lubricant is in the range of about 0.5-3% by weight of the composition.

14. The pharmaceutical composition as claimed in claim 6, wherein the said glidant is selected from the group consisting of talc, silicon dioxide, starch and/or a combination thereof.

15. The pharmaceutical composition as claimed in claim 14, wherein the said glidant is in the range of about 0.5-3% by weight of the composition.

16. The pharmaceutical composition as claimed in claim 6, wherein the said binder is selected from the group consisting of cellulose and its derivatives including, ethyl cellulose, hydroxypropyl cellulose, hydroxypropyl methylcellulose, methylcellulose and hydroxyethyl cellulose, carboxymethyl cellulose; gelatin, liquid glucose; starch; pregelatinized starch; hydrocolloids; sugars; polyvinyl pyrrolidone, sodium alginate, acacia, alginic acid, tragacanth, xanthan, and/or a combination thereof.

17. The pharmaceutical composition as claimed in claim wherein the said binder is in the range of about 1-20% by weight of the composition.

18. The pharmaceutical composition as claimed in claim 1, comprising
a) a therapeutically effective amount of clobazam of defined panicle size or its pharmaceutically acceptable salt(s) or solvate(s),
b) from about 1% to about 30% by weight of pregelatinized starch,
c) from about 40% to about 75% by weight of lactose monohydrate wherein the percentage by weight is relative to total weight of the composition thereof and one or more pharmaceutically acceptable excipient.

19. The pharmaceutical composition according to claim 1, wherein the composition is formulated as a tablet.

20. A process for preparing a pharmaceutical composition containing a therapeutically effective amount of clobazam or its pharmaceutically acceptable salts or solvates thereof comprising the steps of:
(i) micronizing said clobazam until the mean particle size (D₅₀) of said clobazam is equal to or less than about 55μ; (D₉₀) is less than 170 micron; and
(ii) combining said micronized clobazam or its pharmaceutically acceptable salts or solvates with one or more pharmaceutically acceptable excipient;
wherein said micronized clobazam or its pharmaceutically acceptable salts or solvates and said pharmaceutically acceptable excipient are combined by a wet or a dry granulation process.

21. The process according to claim 20, wherein said wet granulation process comprises the steps of:
(i) sifting said micronized clobazam or its pharmaceutically acceptable salts or solvates with at least one diluent, and other excipients to form a mixture;
(ii) granulating the mixture of step (i) with water;
(iii) drying and milling the granulated mixture of step (ii);
(iv) blending the mixture of step (iii) with a at least one filler and
(v) blending the mixture of step (iv) with at least one lubricant; and
(vi) compressing the drug mixture of step (v) into a pharmaceutical dosage form.

22. The process according to claim 20, wherein said dry granulation process comprises the steps of:
(i) micronized clobazam or its pharmaceutically acceptable salts or solvates with one or more pharmaceutically acceptable excipients to form a first mixture;
(ii) granulating and sieving the first mixture of step (i) to the desired size to produce fraction;
(iii) blending the granules of step (ii) with one or more pharmaceutically acceptable excipients to form a dry mixture;
(iv) blending the dry mixture of step win with an extra granular portion, of one or more pharmaceutically acceptable excipients:
(v) blending the mixture of step (iv) with at least one lubricant; and
(vi) compressing the drug mixture of step (v) into a pharmaceutical dosage form;
(vii) optionally coating the pharmaceutical dosage form with a suitable coating material.

23. (canceled)

24. A method for treating tonic-clonic, complex partial or myoclonic seizures; seizures associated with Lennox-Gastaut Syndrome; epilepsy or anxiety comprising administering a composition according to claim 1 to a subject in need thereof.
